(19) United States
(12) Patent Application Publication
(10) Pub. No.: US 2013/0324709 A1
(43) Pub. Date: Dec. 5, 2013

(54) PROCESS FOR THE PREPARATION OF 2-DEOXY-2-FLUORO-2-METHYL-D-RIBOFURANOSYL NUCLEOSIDE COMPOUNDS

(71) Applicant: Hoffmann-La Roche Inc., Nutley, NJ (US)

(72) Inventors: Robert Carr, Effingham, NC (US); Stefan Hildbrand, Gelterkinden (CH); Mark L. Hodges, Florence, NC (US); Michael Kammerer, Basel (CH); John F. Lang, Florence, SC (US); William J. Lawrimore, III, Florence, SC (US); Dieu Nguyen, Wilmington, MA (US)

(21) Appl. No.: 13/903,726

(22) Filed: May 28, 2013

(30) Foreign Application Priority Data
May 29, 2012 (EP) 12169760

(51) Int. Cl. C07H 19/06

(52) U.S. Cl. CPC C07H 19/06
USPC 536/28.4

(57) ABSTRACT

An improved process for the preparation of (2′R)-2′-deoxy-2′-fluoro-2′-methylcytidine derivatives of formula I

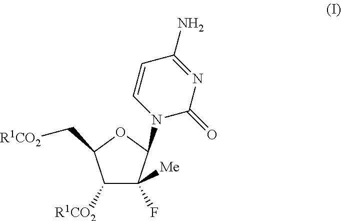

wherein R¹ is selected from C₁₋₄-alkyl is described.

The (2′R)-2′-deoxy-2′-fluoro-2′-methylcytidine derivatives of formula I have the potential to be useful as prodrugs for potent inhibitors of the Hepatitis C Virus (HCV) NS5B polymerase.

### CROSS REFERENCE TO PRIOR APPLICATIONS

This application claims the benefit of priority under 35 U.S.C. §119(a) to EP 12169760.1 filed May 29, 2012 the contents of which is herein incorporated by reference in its entirety.

### BACKGROUND OF THE INVENTION

The present invention relates to an improved process for the preparation of (2′R)-2′-deoxy-2′-fluoro-2′-methylcytidine derivatives of formula I

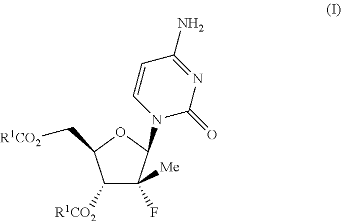

wherein R¹ is selected from C₁₋₄-alkyl which have the potential to be useful as prodrugs for potent inhibitors of the Hepatitis C Virus (HCV) NS5B polymerase (PCT Int. Publ. WO 2007/065829).

### BRIEF SUMMARY OF THE INVENTION

The present invention provides a process for preparing a (2′R)-2′-deoxy-2′-fluoro-2′-methylcytidine derivative of formula I wherein R¹ is selected from C₁₋₄-alkyl, comprising the steps

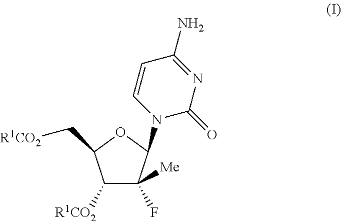

a) transforming a (2R)-2-deoxy-2-fluoro-2-methyl-D-ribonolactone derivative II wherein R² is phenyl or C₁₋₄-alkyl into a (2R)-2-deoxy-2-fluoro-2-methyl-D-ribofuranosyl chloride of formula III wherein R² is phenyl or C₁₋₄-alkyl;

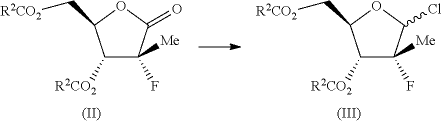

b₁) treating N-benzoyl-cytosine (VIa) with a silylating agent to afford silylated N-benzoyl cytosine VIb; and,

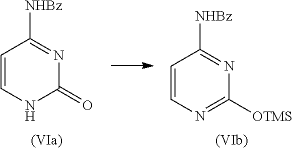

(b₂) coupling VIb with the (2R)-2-deoxy-2-fluoro-2-methyl-D-ribofuranosyl chloride of formula III wherein R² is phenyl or C₁₋₄-alkyl and Bz is benzoyl in the presence of dichloromethane as solvent and a Lewis acid, to form a (2′R)—N-benzoyl-2′-deoxy-2′-fluoro-2′-methyl-cytidine derivative of formula IV wherein R² is phenyl or C₁₋₄-alkyl;

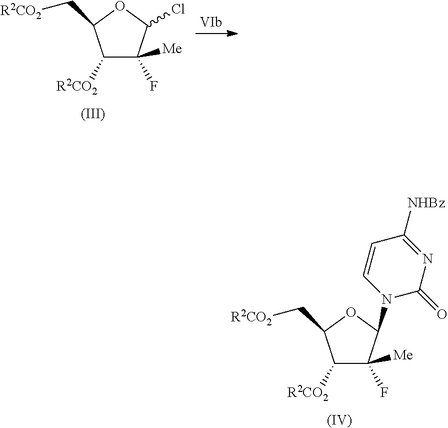

c) alcoholysis of the (2′R)—N-benzoyl-2′-deoxy-2′-fluoro-2′-methyl-cytidine derivative IV to afford (2′R)-2′-deoxy-2′-fluoro-2′-methyl-cytidine (V); and,

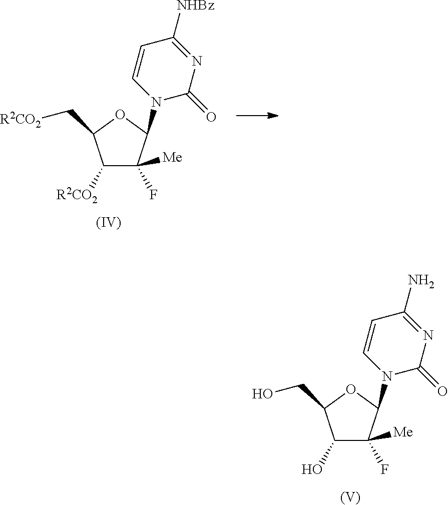

d) acylating (2′R)-2′-deoxy-2′-fluoro-2′-methyl-cytisdine (V) to form the (2′R)-2′-deoxy-2′-fluoro-2′-methylcytidine derivative of formula I.

### DETAILED DESCRIPTION OF THE INVENTION

The preparation of the (2′R)-2′-deoxy-2′-fluoro-2′-methylcytidine derivatives of formula I can follow the known steps:

a) transforming the (2R)-2-deoxy-2-fluoro-2-methyl-D-ribonolactone derivative of formula II wherein R² is phenyl or C₁₋₄-alkyl into the (2R)-2-deoxy-2-fluoro-2-methyl-D-ribofuranosyl chloride of formula III wherein R² is phenyl or C₁₋₄-alkyl;

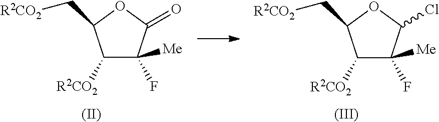

b) coupling the (2R)-2-deoxy-2-fluoro-2-methyl-D-ribofuranosyl chloride of formula III with N-benzoyl cytosine of formula VIa to form the (2′R)—N-benzoyl-2′-deoxy-2′-fluoro-2′-methyl-cytidine derivative of formula IV wherein R² is as above and Bz is benzoyl;

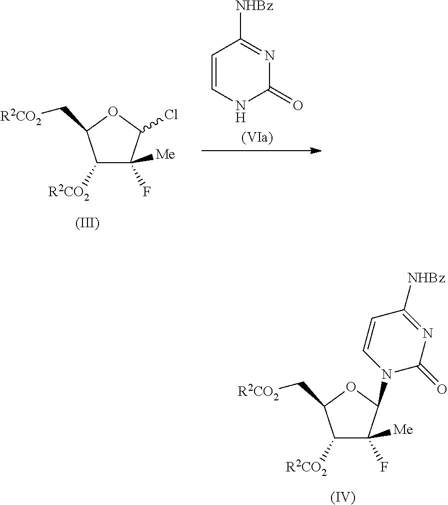

c) alcoholysis of (2′R)—N-benzoyl-2′-deoxy-2′-fluoro-2′-methyl-cytidine derivative of formula IV to afford the (2′R)-2′-deoxy-2′-fluoro-2′-methyl-cytidine of formula;

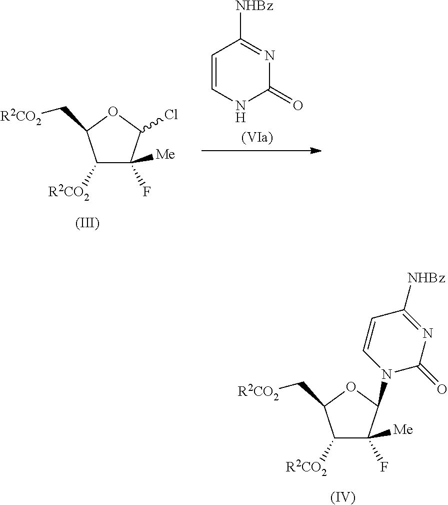

and finally

d) acylating the (2′R)-2′-deoxy-2′-fluoro-2′-methyl-cytidine of formula V to form the (2′R)-2′-deoxy-2′-fluoro-2′-methylcytidine derivative of formula I.

It was found that the crucial step for a technical scale synthesis of the (2′R)-2′-deoxy-2′-fluoro-2′-methylcytidine derivatives of formula I is the coupling step b). According to PCT Int. Appl. WO 2008/045419 this step requires the use of substantial amounts of chlorobenzene as solvent. The corrosive nature of this solvent renders it undesirable for large scale processes. It was further observed that the quench produces exothermy and HCl release which are difficult to control. In addition the subsequent filtration of the precipitated excess of N-benzoyl cytosine wase very slow and accordingly resulted in a limiting factor regarding scale up capacity. The object of the present invention therefore was to improve synthesis step b) in such a manner that the process can be applied on technical scale.

The object of the invention could be achieved with the process of the present invention which comprises the preparation of the (2′R)-2′-deoxy-2′-fluoro-2′-methylcytidine derivative of formula I wherein R¹ is selected for C₁₋₄-alkyl comprising the steps:

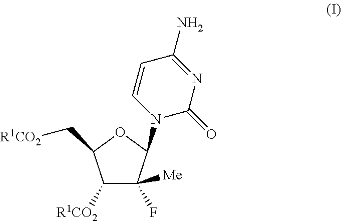

a) transforming the (2R)-2-deoxy-2-fluoro-2-methyl-D-ribonolactone derivative of formula II wherein R² is phenyl or C₁₋₄-alkyl into the (2R)-2-deoxy-2-fluoro-2-methyl-D-ribofuranosyl chloride of formula III wherein R² is phenyl or C₁₋₄-alkyl;

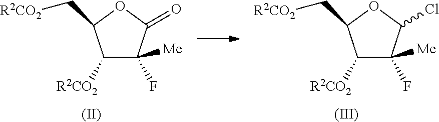

b₁) silylating of N-benzoyl cytosine (VIa) to form the silylated N-benzoyl cytosine of formula VIb; in the presence of a C₃₋₄-alkylacetate as solvent;

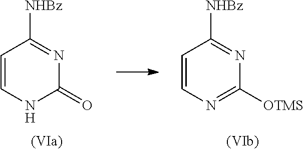

b₂) the coupling of the silylated N-benzoyl cytosine (VIb) with the (2R)-2-deoxy-2-fluoro-2-methyl-D-ribofuranosyl chloride of formula III wherein R² is phenyl or C₁₋₄-alkyl, in the presence of dichloromethane as solvent and a Lewis acid to form the (2′R)—N-benzoyl-2′-deoxy-2′-fluoro-2′-methyl-cytidine derivative of formula IV wherein R² is as above and Bz is benzoyl.

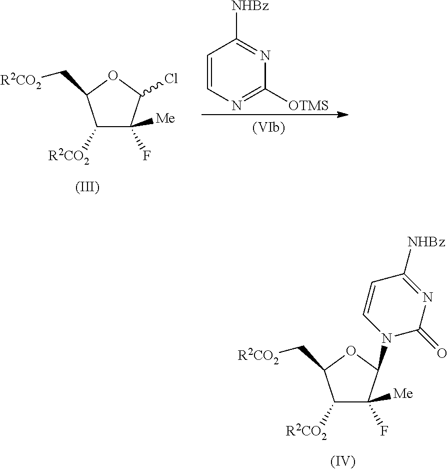

c) alcoholysis of the (2′R)—N-benzoyl-2′-deoxy-2′-fluoro-2′-methyl-cytidine derivative of formula IV to afford the (2′R)-2′-deoxy-2′-fluoro-2′-methyl-cytidine of formula V and;

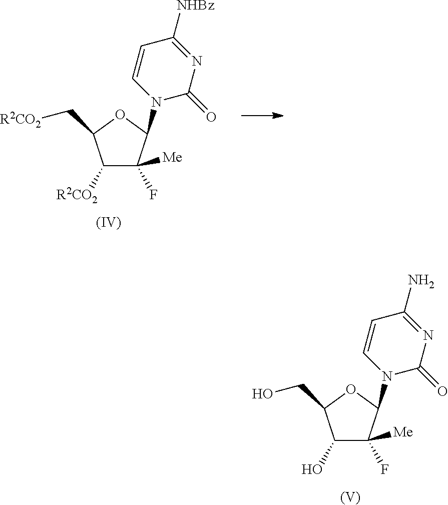

d) acylating the (2′R)-2′-deoxy-2′-fluoro-2′-methyl-cytidine of formula V to form the (2′R)-2′-deoxy-2′-fluoro-2′-methylcytidine derivative of formula I,

The following definitions are set forth to illustrate and define the meaning and scope of the various terms used to describe the invention herein.

The term “C₁₋₄-alkyl” as used herein denotes an unbranched or branched chain, saturated, monovalent hydrocarbon residue containing 1 to 4 carbon atoms, particularly methyl, ethyl, n-propyl, i-propyl, n-butyl, sec-butyl or t-butyl.

The term “C₃₋₄-alkyl” as used herein denotes an unbranched or branched chain, saturated, monovalent hydrocarbon residue containing 3 to 4 carbon atoms, particularly n-propyl, i-propyl, n-butyl, sec-butyl or t-butyl, more particularly i-propyl or n-butyl.

Step a)

The transformation in step a) comprises a reduction in the presence of a reducing agent and a subsequent chlorination in the presence of chlorinating agent.

The reducing agent bis-(2-methoxyethoxy) (2,2,2,-trifluoro ethoxy) aluminum hydride is typically preformed from sodium bis-(2-methoxyethoxy) aluminum hydride, which is commercially available under the trade name Red-Al (Vitride®, solution in toluene) and trifluoroethanol.

The reduction usually takes place in an organic solvent such as in toluene at a reaction temperature of 0° C. to −30° C.

After completion of the reduction the reaction mixture is subjected to the chlorination reaction. The chlorinating agent is as a rule selected from sulfuryl chloride, thionyl chloride or phosphorus oxychloride. Preferably sulfuryl chloride in the presence of catalytic amounts of tetrabutyl ammonium bromide is used. The addition of the chlorinating agent as a rule takes place at a temperature of −20° C. to 0° C., thereafter the reaction temperature is maintained between 20° C. and 30° C.

The (2R)-2-deoxy-2-fluoro-2-methyl-D-ribofuranosyl chloride of formula III can be separated from the reaction mixture applying techniques known to the skilled in the art.

Step b)

Coupling step b) is characterized by the steps

b₁) the silylation of N-benzoyl cytosine (VIa) to form the silylated N-benzoyl cytosine of formula VIb in the presence of a C₃₋₄-alkylacetate as solvent; and,

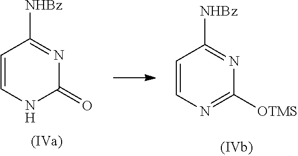

b₂) coupling (VIb) with the (2R)-2-deoxy-2-fluoro-2-methyl-D-ribofuranosyl chloride of formula III, wherein R² is phenyl or C₁₋₄-alkyl, to form the (2′R)—N-benzoyl-2′-deoxy-2′-fluoro-2′-methyl-cytidine derivative of formula IV, wherein R² is as above and Bz is benzoyl, in the presence of dichloromethane as solvent and a Lewis acid.

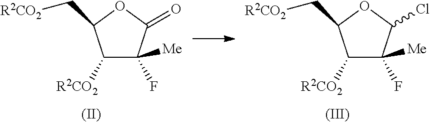

Step b₁

The silylation can be performed with a suitable silylating agent such as with hexamethyldisilazane usually in the presence of ammonium sulfate. Suitable C₃₋₄-alkylacetate solvents are i-propyl or n-butyl acetate. The reaction typically takes place at temperatures higher than 85° C., i.e. particularly at the reflux temperature of the solvent, for about 3 h to 8 h. The resulting solution of the silylated N-benzoyl cytosine of formula VIb can be concentrated and used directly in the subsequent reaction step b₂).

Step b₂

For step b₂) the former solvent is completely exchanged with dichloromethane. Common Lewis acids known in the art are suitable for the conversion in step b₂). Particularly good results have been achieved with tin tetrachloride. The reaction is usually performed at a reaction temperature of 70° C. to 90° C. and a pressure of 2 bar to 3 bar. More particularly the reaction temperature is 75° C. to 85° C. and at a pressure of 2.5 bar.

In a further particular embodiment the reaction mixture, after completion of the coupling reaction in step b₂), is quenched by adding it to a mixture of acetic acid and water of 97:3 (w/w) to 80:20 (w/w), more particularly of 95:5 (w/w) to 90:10 (w/w), at a temperature of 10° C. to 30° C., more particularly at a temperature of 15° C. to 25° C.

In a further particular embodiment the (2′R)—N-benzoyl-2′-deoxy-2′-fluoro-2′-methyl-cytidine derivative of formula IV, so obtained in step b₂) can be further purified by multiple extractions of the tin with a mixture of water and acetic acid and subsequent crystallization after partly replacing the dichloromethane by methanol.

The ratio of water and acetic acid in the mixture expediently is 1 to 3:1 (v/v). The extractions are repeated until the tin content in the isolated product is reproducibly <20 ppm. As a rule this target can be reached with 3 to 4 extraction cycles. The ratio of methanol and dichloromethane in the mixture for the crystallization is usually 2 to 5:1 (w/w).

Step c)

The alcoholysis in step c) is performed in the presence of a base and an alcohol as solvent. Suitable bases are organic bases like alkali metal alkoxides, particularly sodium methoxide. In a particular embodiment 0.03 eq. to 0.10 eq. sodium methoxide in methanol is used. The alcoholysis reaction is usually performed at a reaction temperature of 50° C. to 65° C.

When the alcoholysis is completed, the (2′R)-2′-deoxy-2′-fluoro-2′-methyl-cytidine (V) can typically be separated from the reaction mixture by applying techniques known to the skilled in the art, for instance by crystallization from isopropanol/methanol.

Step d)

The acylation in step d) is as a rule performed with a C₁₋₄-alkanoyl chloride in the presence of an organic solvent/water mixture at temperatures of −5° C. and 5 C. In a particular embodiment isobutyryl chloride is the selected C₁₋₄-alkanoyl chloride and tetrahydrofuran is the selected organic solvent. The isolation of the (2′R)-2′-deoxy-2′-fluoro-2′-methylcytidine derivative of formula I from the reaction mixture can follow methods known to the skilled in the art, for instance by an extraction of the neutralized reaction mixture with ethyl acetate and subsequent crystallization in a mixture of a C₁₋₄-alcohol and n-heptane. Suitable C₁₋₁₄-alcohols are methanol, ethanol and i-propanol. In a particular embodiment the crystallization is performed with a mixture of i-propanol and n-heptane of 3:7 (v/v).

### EXAMPLES

The abbreviations used include: dichloromethane (DCM), 4-N,N-dimethylaminopyridine (DMAP), hexamethyldisilazane (HMDS), ethanol (EtOH), ethyl acetate (AcOEt), methanol (MeOH), methyl (Me), ethyl (Et), isopropanol, phenyl (Ph), benzoyl (Bz), room temperature (rt or RT), triethylamine (TEA or Et₃N), tetrahydrofuran (THF) and trimethylsilyl (TMS).

### Example 1

### Step a: Preparation (2R)-2-deoxy-2-fluoro-2-methyl-α/β-D-erythro-pentofuranosyl chloride-3,5-dibenzoate

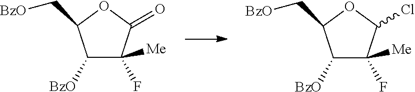

A solution of 132 g of trifluoroethanol in 110 g of toluene was slowly added at −30 to −10° C. to a solution of 381 g of Red-Al (Vitride®, 66.5% solution in toluene) in 90 g of toluene and the stirring the resulting mixture for 30 minutes. The mixture was then allowed to warm to room temperature where it can be stored for several weeks.

114.7 g of this modified Red-Al reagent was added within 2 to 3 hours to a suspension of 60 g of (2R)-2-deoxy-2-fluoro-2-methyl-D-ribonolactone-3,5-dibenzoate in 108 g of toluene and 78 g of butyl acetate while maintaining the temperature between −15 to −20° C. and the resulting mixture was stirred for 1 to 2 hours. When the reaction was complete, 0.6 g of tetrabutylammonium bromide was added. The solution was cooled to −20 to 0° C. then 75.0 g of sulfuryl chloride was added over 1 hour. After addition completion, the mixture was warmed to 17 to 20° C. and hold at this temperature for 4 to 5 hours. The reaction mixture was then quenched by adding it at 15 to 40° C. to a preformed solution of 180 g of sodium citrate dihydrate in 420 g of water. The first reactor and the transfer lines were rinsed with 60 g of butyl acetate. 38 g of sodium hydroxide (42% in water) was then added and the biphasic mixture was stirred for 1 hour at 30-35° C. The layers were allowed to settle for at least 30 minutes and the lower aqueous phase was removed. The organic layer was washed at 28-35° C. with first an aqueous solution of 60 g of sodium citrate dihydrate in 140 g of water, followed by 200 g of water. From the organic layer water, toluene and butyl acetate were distilled off at a maximum temperature of 50° C. and replaced by isopropyl acetate to afford 301.5 g of an isopropyl acetate solution containing 18.0% (w/w) of the title compound as an α/β-anomeric mixture (86% yield) which was used without further purification in the subsequent step b).

### Step b: Preparation of (2′R)—N-benzoyl-2′-deoxy-2′-fluoro-2′-methyl-cytidine-3′,5′-dibenzoate

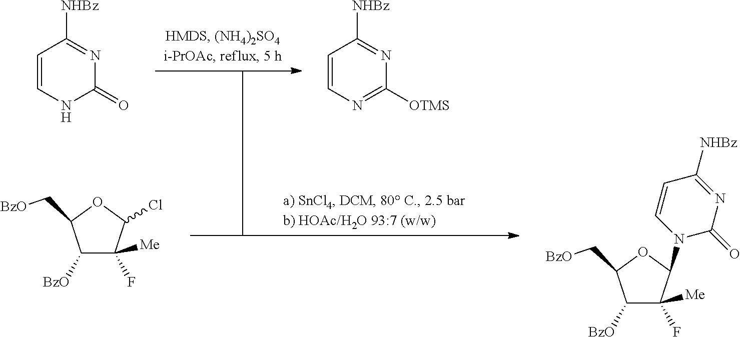

To a suspension of N-benzoyl cytosine (30.2 g, 140.3 mmol) and ammonium sulfate (400 mg, 2.8 mmol) in isopropyl acetate (320 mL) was added at reflux temperature over 30 to 60 minutes hexamethyldisilazane (22.5 g, 139.4 mmol) and the resulting mixture was stirred at reflux temperature (88-90° C.) until a clear solution was obtained (approx. 5 hours). The solution was then concentrated under reduced pressure at approx. 40° C. to a residual volume of approx. 90 mL. then 200 g of (2R)-2-deoxy-2-fluoro-2-methyl-α/β-D-erythro-pentofuranosyl chloride-3,5-dibenzoate (18% (w/w) solution in isopropyl acetate; 91.3 mmol) was added and the resulting mixture concentrated under reduced pressure at approx. 40° C. to a residual volume of approx. 90 mL. The residue was treated with 200 mL of n-heptane and the solvents were completely removed at approximately 40° C. to yield a viscous oil. The oil was diluted with 340 mL of dichloromethane and the resulting turbid solution was treated at >30° C. with tin tetrachloride (46.4 g, 178.1 mmol). The reactor was closed and the resulting mixture heated to 75-80° C. (−2.5 bars). The mixture was stirred at this temperature for 20 hours and subsequently cooled to room temperature. The reaction mixture was then added over 1 to 2 hours at 18 to 25° C. to a preformed mixture of 72 g of acetic acid and 5.4 g water and the obtained grey suspension was subsequently stirred at 22° C. for an additional hour. The suspension was filtered and the transfer lines and the filter cake were washed in portions with 160 mL of dichloromethane. To the filtrate water (170 mL) and acetic acid (170 mL) were added and the biphasic mixture was stirred for 20 minutes at 30° C. The layers were then allowed to separate for 30 minutes. The lower organic phase was separated and subsequently washed three times with a mixture of water (150 mL) and acetic acid (50 mL). The organic layer was then polish filtered (using a ZetaCarbon™ filter cartridge). The filtrate was diluted with 300 mL of methanol and from the resulting mixture dichloromethane/methanol was distilled off at atmospheric pressure and the removed solvent was continuously replaced by methanol keeping the volume in the reactor constant at 850-900 mL. The distillation was stopped when the batch temperature was 52° C. The resulting suspension was cooled to 20° C. within 3 hours and stirred at this temperature for 2 hours. The crystals were filtered off, washed with methanol (200 mL) and dried at 55° C./<10 mbar to afford 34.9 g (67% yield) of the title compound with an assay (HPLC) of 99.8% (w/w) and a tin content of 9 ppm.

### Step c: Preparation of (2′R)-2′-deoxy-2′-fluoro-2′-methylcytidine

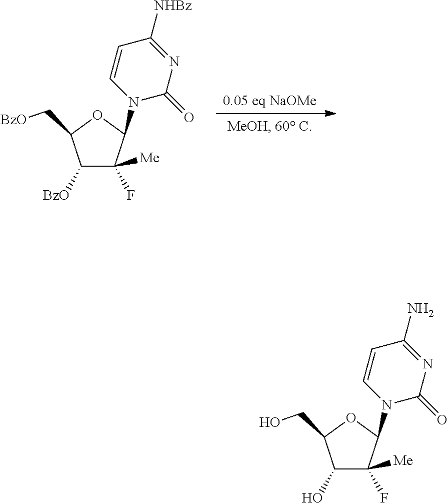

To a suspension of (2′R)—N-benzoyl-2′-deoxy-2′-fluoro-2′-methyl-cytidine-3′,5′-dibenzoate (62.1 g, 108.6 mmol) in methanol (420 mL) at 60° C. was added sodium methoxide (25% (w/w) in methanol, 1.17 g, 5.4 mmol, 0.05 eq) and the resulting suspension was stirred at 60° C. for 4 hours. Isobutyric acid (0.58 g, 6.5 mmol, 0.06 eq) was then added and the resulting mixture was polish filtered. Methanol was distilled from the filtrate at atmospheric pressure and the distilled solvent was continuously replaced by isopropanol maintaining the volume in the reactor constant at −300 mL. In total, 400 mL of isopropanol was added during the solvent exchange. The resulting suspension was cooled from 80 to −2° C. within 5 hours and subsequently stirred at this temperature for 4 hours. The crystals were filtered off, washed with isopropanol and dried at 70° C./<10 mbar to afford 25.6 g (91% yield) of the title compound with an assay (HPLC) of 99.6% (w/w).

### Step d: Preparation of (2′R)-2′-deoxy-2′-fluoro-2′-methylcytidine-3′,5′-diisobutyrate (Mericitabine)

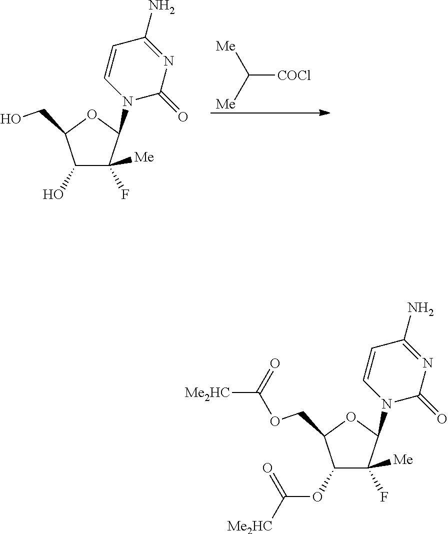

### Example 1

In a jacketed vessel 20.0 g of (2′R)-2′-deoxy-2′-fluoro-2′-methylcytidine, 1.08 g of DMAP, and 54.4 g of TEA were suspended in 82.4 g of water and 184 g of THF, the mixture was cooled to 0±5° C. and 40 g of isobutyryl chloride were added within 1 to 2 hours while maintaining the temperature at 0±5° C. When the addition was complete, the solution was warmed to room temperature and the pH was adjusted to pH 6.0 to 7.0 with conc. hydrochloric acid. 120 g of ethyl acetate were then added and the biphasic mixture stirred for 20 minutes. The layers were allowed to separate for 20 minutes. The aqueous layer was separated (and discarded) and the organic layer was washed first with a mixture of 56 g of saturated aqueous sodium bicarbonate solution and 38 g of water followed by 72 g of water. The organic layer was concentrated to a volume of <50 mL under vacuum with a jacket temperature 50 to 70° C. 325 g of isopropanol were charged in portions while the solution was concentrated under vacuum. A total of 250 g of isopropanol was distilled from the vessel. The mixture was heated to 70-75° C. and 275 g of n-heptane were added at this temperature within 3 to 4 hours. The resulting suspension was then cooled to −5 to 0° C. within 6 hours. After 2 hours stirring at this temperature, the crystals were filtered off, washed with a mixture of 10 g isopropanol and 30 g of n-heptane and dried at 50±+5° C./<10 mbar to afford 26.7 g (86% yield) of the title compound with an assay (HPLC) of 99.3% (w/w).

### Example 2

In a 1000 mL double jacket reactor 40.0 g of (2′R)-2′-deoxy-2′-fluoro-2′-methylcytidine, 0.12 g of DMAP, and 98 g of TEA were added at 40° C. to a mixture of 160 g of water and 350 g of THF. The resulting solution was cooled to 0±+5° C. and 68 g of isobutyryl chloride were added within 4 to 5 hours while maintaining the temperature at 0±+5° C. When the addition was complete, the solution was stirred for one additional hour at 0° C. The pH was then adjusted to pH 6.0 to 7.0 with 20% aqueous sulfuric acid while maintaining the reaction temperature at 0° C. 150 g of ethyl acetate were added and the biphasic mixture stirred for 20 minutes at 0° C. The layers were allowed to separate for 20 minutes. The aqueous layer was separated (and discarded) and the organic layer was treated with 100 g of water and the pH of the mixture was adjusted to pH 10.5 to 11.0 with 28% aqueous sodium hydroxide while the temperature was maintained at 0° C. Ethyl acetate (110 g) was added and the biphasic mixture was allowed to warm to rt and stirred at this temperature for 2 hours. The layers were allowed to separate for 20 minutes. The aqueous layer was separated (and discarded). The organic layer was washed once with diluted aqueous sulfuric acid (110 g) and then with water (50 g). From the organic layer, ethyl acetate, THF, and water were completely removed by distillation and replaced by isopropanol. The resulting mixture (containing approx. 17% (w/w) of the title compound) was heated to 65-70° C. and 130 g of n-heptane were added at this temperature within 30 minutes. After seeding, the mixture was cooled to 55° C. within 3 to 5 hours and 170 g of n-heptane were added at this temperature within one hour. The resulting suspension was then cooled to 0° C. within 3 to 5 hours. At this temperature additional 600 g of n-heptane were added within one hour and the suspension stirred for 2 hours. The crystals were filtered off, washed with 150 g of n-heptane and dried at 50±+5° C./<10 mbar to afford 55.2 g (90% yield) of the title compound with an assay (HPLC) of 99.4% (w/w).

1. A process for the preparation of a (2′R)-2′-deoxy-2′-fluoro-2′-methylcytidine derivative of formula I wherein R¹ is selected from C₁₋₄-alkyl, comprising the steps 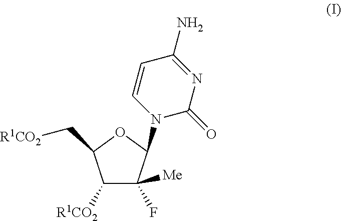
a) transforming a (2R)-2-deoxy-2-fluoro-2-methyl-D-ribonolactone derivative (II) wherein R² is phenyl or C₁₋₄-alkyl into a (2R)-2-deoxy-2-fluoro-2-methyl-D-ribofuranosyl chloride of formula III wherein R² is phenyl or C₁₋₄-alkyl; 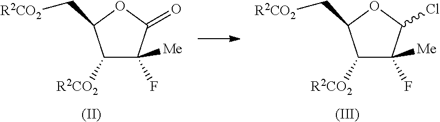
b₁) treating N-benzoyl-cytosine (VIa) with a silylating agent to afford silylated N-benzoyl cytosine (VIb); and, 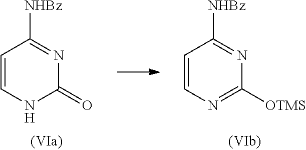
(b₂) the coupling VIb with the (2R)-2-deoxy-2-fluoro-2-methyl-D-ribofuranosyl chloride of formula III wherein R² is phenyl or C₁₋₄-alkyl and Bz is benzoyl, in the presence of dichloromethane as solvent and a Lewis acid, to form a (2′R)—N-benzoyl-2′-deoxy-2′-fluoro-2′-methyl-cytidine derivative of formula IV wherein R² is phenyl or C₁₋₄-alkyl; 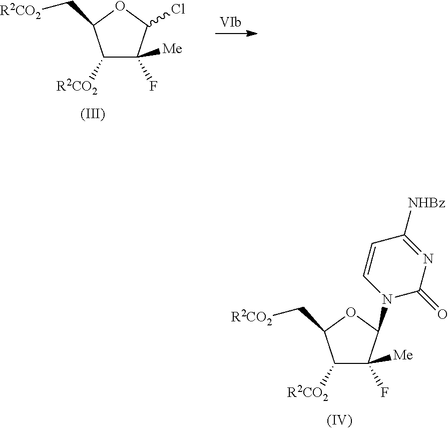
c) alcoholysis of the (2′R)—N-benzoyl-2′-deoxy-2′-fluoro-2′-methyl-cytidine derivative (IV) to afford (2′R)-2′-deoxy-2′-fluoro-2′-methyl-cytidine (V); and, 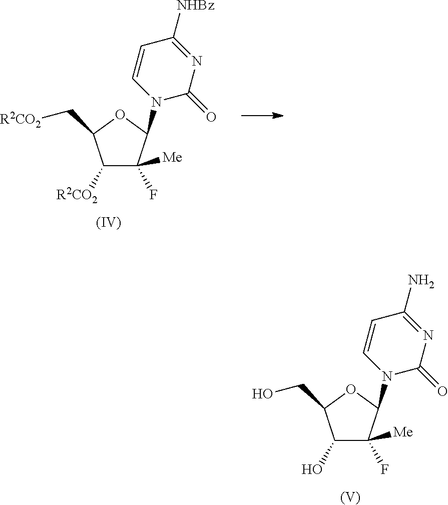
d) acylating (2′R)-2′-deoxy-2′-fluoro-2′-methyl-cytisdine (V) to form the (2′R)-2′-deoxy-2′-fluoro-2′-methylcytidine derivative of formula I.

2. The process according to claim 1 wherein R² is phenyl.

3. The process according to claim 1 wherein R¹ is i-propyl.

4. The process according to claim 1 wherein that i-propyl- or n-butyl acetate is used as solvent for step b₁).

5. The process according to claim 4 wherein the silylation in step b₁) is performed with hexamethyldisilazane in the presence of ammonium sulfate.

6. The process according to claim 1 wherein the Lewis acid used in step b₂) is tin tetrachloride.

7. The process according to claim 6 wherein the coupling in step b₂) is performed at a reaction temperature of 70° C. to 90° C. and a pressure of 2 bar to 3 bar.

8. The process according to claim 7 wherein the reaction mixture, after completion of the coupling reaction in step b₂), is quenched with a mixture of acetic acid and water of 97:3 (w/w) to 80:20 (w/w) at a temperature of 10° C. to 30° C.

9. The process according to claim 8 wherein the (2′R)—N-benzoyl-2′-deoxy-2′-fluoro-2′-methyl-cytidine derivative of formula IV obtained in step b₂) is further purified by multiple extractions of the tin with a mixture of water and acetic acid and subsequent crystallization by replacing partly of the dichloromethane by methanol.

10. The process according to claim 9 wherein the ratio of water and acetic acid for the extraction is 1 to 3:1 (v/v) and the ratio of methanol and dichloromethane for the crystallization is 2 to 5:1 (w/w).

11. The process according to claim 1 wherein the transformation in step a) comprises a reduction in the presence of a reducing agent and a subsequent chlorination in the presence of chlorinating agent.

12. The process according to claim 11 wherein the reducing agent is preformed from sodium bis-(2-methoxyethoxy) aluminum hydride und trifluoroethanol.

13. The process according to claim 11 wherein the chlorinating agent is selected from sulfuryl chloride, thionyl chloride or phosphorus oxychloride.

14. The process according to claim 13 wherein the chlorinating agent is sulfuryl chloride in the presence of catalytic amounts of tetrabutylammonium bromide.

15. The process according to claim 1 wherein the alcoholysis in step c) is performed in the presence of a base and an alcohol as solvent.

16. The process according to claim 15 wherein the base is sodium methoxide and the organic solvent is methanol.

17. The process according to claim 16 wherein 0.03-0.10 equiv. of sodium methoxide is used at a reaction temperature of 50° C. to 65° C.

18. The process according to claim 1 wherein the acylation in step d) is performed with a C₁₋₄-alkanoyl chloride in the presence of an organic solvent/water mixture at temperatures of −5° C. and 5° C.

19. The process according to claim 18 wherein the C₁₋₄-alkanoylchloride is isobutyryl chloride and the organic solvent is tetrahydrofuran.

20. The process according to claim 18 wherein the (2′R)-2′-deoxy-2′-fluoro-2′-methylcytidine derivative of formula I obtained from step d) is crystallized in a mixture of a C₁₋₄-alcohol and n-heptane.
